(19) United States
(12) Patent Application Publication
(10) Pub. No.: US 2002/0103349 A1
(43) Pub. Date: Aug. 1, 2002

(54) Drug-oligonucleotides chimeric molecules

(76) Inventor: Asher Nathan, Palo Alto, CA (US)

Correspondence Address: James E. Eakin, McDermott, Will & Emery, 3150 Porter Drive, Palo Alto, CA, 94304-1212, US

(21) Appl. No.: 09/865,987

(22) Filed: May 25, 2001

Related U.S. Application Data
(60) Provisional application No. 60/206,959, filed on May 25, 2000.
(60) Provisional application No. 60/207,369, filed on May 26, 2000.
(60) Provisional application No. 60/207,399, filed on May 30, 2000.
(60) Provisional application No. 60/232,615, filed on Sep. 14, 2000.
(60) Provisional application No. 60/259,231, filed on Jan. 3, 2001.

(51) Int. Cl. C07H 21/02; C07H 21/04

(52) U.S. Cl. 536/23.1

(57) ABSTRACT

The present invention relates to methods of using oligonucleotides for increasing the efficacy of existing drugs. In some embodiments, oligonucleotides are selected from a random population for their ability to enter cells. Then the selected oligonucleotides are combined with existing drugs to form chimeric-drug oligonucleotide molecules that access cells more readily than the uncombined drugs. Alternatively, the oligonucleotides are combined with the existing drug, and the resulting chimeric molecules are selected for their ability to enter cells. The chimeric drug-olignucleotide molecules are designed to improve various pharmacological properties of existing drugs, including organ and tissue specificity, and drug targeting.

Provisional No. 60/259,231, filed Jan. 2, 2001, title: Drug-Oligonucleotides Chimeric Molecules; and to co-pending US patent applications filed on even date herewith, the first of which is entitled “Extra Cellular Drug-Oligonucleotides Chimeric Molecules”, S/N:______, attorney docket no. 57557-014, and the second of which is entitled “Drug-Amino Acids Chimeric Molecules”, S/N:______, attorney docket no. 57557-013, both incorporated herein by reference.

### FIELD OF THE INVENTION

The present invention relates to methods of increasing the efficacy of existing drugs, and more particularly relates to modifying known drugs by combining them with oligonucleotides to produce chimeric drug-oligonucleotide molecules having superior targeting, uptake and retention than the unmodified known drug.

### BACKGROUND OF THE INVENTION

Pharmaceutical and biotechnology companies currently select and optimize the majority of preclinical drug candidates based on their in vitro characteristics. Yet, in order for a drug to pass through all the regulatory hurdles needed to become an approved compound, it must also possess, by coincidence, the relevant in vivo characteristics. In vitro screens are based on the ability of each drug candidate to interact with a specific molecular target. The reason that most candidate drugs are not effective in the body is that their true pharmacokinetic properties cannot be adequately assessed in vitro.

Few lead compounds survive the preclinical and clinical trial processes. Each year an average pharmaceutical company will screen nearly 5,000 compounds as possible candidates for new medicines. Only about 5%, or approximately 250 of the initially screened compounds, will survive to the preclinical laboratory testing stage. Out of the 250 compounds that successfully completed pre-clinical trial testing on the order of five candidates, or 0.1% of the original 5,000 compounds, will pass the regulatory hurdles required to begin clinical trials in human subjects. Of the five potential therapeutics that any given company might have identified, only one of them will make it through to completion of phase III clinical trials and then be approved by the Food and Drug Administration (Source: IMS Health). These statistics highlight the gap between a drug's activity in a test tube and its efficacy inside the body, and point to the inadequacies of in vitro selected drugs to complete successfully the preclinical and clinical developmental phases.

Many aspects of normal human physiology account for the reasons that successful in vitro lead compounds fail in vivo. The body prevents the drug from interacting with the target through a variety of mechanisms that involve absorption, distribution, metabolism, and excretion. For example, because most drugs travel to their target sites in a relatively nonspecific manner, sufficient quantities of the drug might never reach their intended sites in order to effect physiological change. Instead, drugs often become concentrated in healthy tissues and organs where they can cause damage. Although current drug discovery tools such as high-throughput screening (HTS) and rational drug design methodologies can select for improved in vitro activity, they are not generally useful for improving the drug's efficacy inside the body. To make changes in a drug candidate's in vivo characteristics, generally requires that medicinal chemists alter the drug's chemistry and then laboriously test each changed molecule, one at a time, in animal models. Due to the high cost and labor-intensive efforts needed to test each drug candidate in an individual animal, most drugs that fail during in vivo testing are discarded.

The inability to efficiently and accurately predict, let alone influence the outcome of a lead compound's behavior in vivo, negatively affects the time, cost, and level of risk associated with the drug development process. The average cost to develop a new drug, from the discovery phase through approval, is estimated to be $500 million dollars and the process takes an average of ten to twelve years to complete. In addition to the high costs and long development times of drugs, the risk in drug development is extraordinarily high because the vast majority of preclinical candidates fail to become drugs. Even successful drugs that have gained regulatory approval generally have not been optimized for their in vivo characteristics and are thus prime candidates for improvement

One factor that has increased the need for high-throughput in vivo optimization is the output from the Human Genome Project and the therapeutic drugs that it will generate. The Genome Project has created an explosion of potential targets, and by extension, the development of new drugs. In the history of drug discovery and development to date, all existing therapeutics has targeted fewer than 500 proteins, such as receptors, enzymes and ion channels. In contrast, it is now estimated that knowledge of the human genome will create an additional 10,000 to 60,000 new molecular targets, which should result in the development of many new drugs. Existing methods, including the newer versions of ultra high throughput screening (UHTS) are unlikely to efficiently screen the multitude of new lead candidates and yield approved drugs.

Typically, attempts at improving existing drugs reside in chemically modifying the drug or, where the drug is the product of genetic techniques, modifying the sequence encoding the drug. Recent molecular techniques have made it feasible to simulate evolutionary processes and apply in vitro evolution to evolve molecules with novel properties that may have potential benefits for medical and industrial applications. In vitro evolution is a process of molecular discovery that mirrors the evolution of organisms in nature. In natural evolution, each organism contains a different DNA sequence, which is the genetic blueprint from which the organism is created. The DNA blueprint is continuously subjected to natural selection. Selection occurs through a process that has been described as survival of the fittest. Organisms that survive selection can pass on a portion of their DNA blueprint to their offspring. The offspring are themselves subjected to further rounds of selection and reproduction so that over time, there is an enrichment of the DNA sequences that impart improved survival qualities. In vitro evolution accelerates the process of natural selection.

To initiate the process of in vitro evolution in the laboratory, an initial population of randomly generated DNA molecules is synthesized. This random population is easily constructed using a conventional, commercially available nucleic acid synthesizer. The initial population of nucleic acid molecules is subjected to an artificial selection pressure whereby the molecules that have the desired behavior in vivo are retained and separated from the rest of the initial population, which is discarded. Rare DNA molecules that have the desired traits, as well as molecules that do not have the desired traits but due to the inaccuracy of the selection process have survived by chance, are amplified in vitro through application of an enzymatic process that exponentially amplifies DNA. Following amplification, the desired population is subjected to iterative rounds of selection and amplification, such that DNA molecules having the desired traits become so sufficiently enriched so that they may be identified using conventional screening techniques.( Tuerk, C., and Gold, L. (1990) Science, 249 (4968), 505-510.)

The first application of in vitro evolution was to evolve molecular diversity of mammalian antibodies (McAfferty, J. et al, (1990) Nature, 348 (6301), 552-554; Kang, A. S. et al, (1991) Proc. Natl. Acad. Sci. USA, 88 (10), 4363-4366). More recently, the application of in vitro evolution of nucleic acids in combination with the technique of phage display has been used to generate a variety of peptides which are screened in vivo to identify the translated sequences that specifically target certain organs (U.S. Pat. No. 5,622,699). Use of these techniques have led to the isolation of peptides that efficiently target molecules from blood to specific locations in the body, including brain, kidney, lung, skin and pancreas (Rajotte, D. et al (1998) J. Clin. Invest. 1021(2), 430-437; Pasqualini, R., and Ruoslahti, E. (1996) Nature, 280, 363-365.). Small peptide moieties, called signal peptides, have also been identified and shown to mediate transport and targeting of large and diverse molecules between the nucleus and cytoplasm of cells, entry into subcellular compartments, secretion from cells, and uptake of molecules into cells from the surrounding fluid. In many cases, the relatively short signal peptide is sufficient to direct virtually any drug molecule to its destination. A striking example is a peptide derived from the Tat protein of HIV, which mediates very efficient entry of attached proteins into cells.

However, nucleic acid sequences are not only informational molecules, but may have additional physiological properties conferred on them by their three-dimensional structure, by their charge, or by their capacity to interact with other nucleic or non-nucleic acid molecules. (Hermann, T., and Patel, D. J. Science, 287, 820-825; U.S. Pat. No. 4,987,071). Relatively short oligonucleotides possess structural diversity. Therefore, within a sufficiently comprehensive collection of such molecules, there will be members that can mimic the simple structures favored by nature for molecular addressing. Recognizing the ability of oligonucleotides to form a multitude of three-dimensional structures, Systematic Evolution of Ligands by Exponential Enrichment (SELEX™) was developed. SELEX™, is a combinatorial chemistry process that applies in vitro evolution to a very large pool of random sequence molecules to identify nucleic acid sequences that have the highest affinity for a variety of proteins and low molecular weight targets (Morris, K. N. et al (1998) Proc. Nati. Acad. Sci. USA, 96, 2902-2907). The SELEX method is described in the following U.S. Pat. No. 5,270,163, 5,475,096, 6,011,020, 5,637,459, 5,843,701 and 5,683,867, which are incorporated herein by reference. One of these patents, U.S. Pat. No. 6,011,020, discloses a method for producing chimeric molecules which comprise a nucleic acid region and a chemically reactive functional unit, wherein the nucleic acid region has a binding activity with the target, and the chemically reactive functional unit is a photoreactive group, an active-site directed compound, or a peptide. As in the other Gold patents the nucleic acid sequence is selected for its specific affinity for binding to a variety of molecular targets. High affinity RNA ligands have also been identified (Homann, M, and Goringer, H. U. (1999) Nucleic Acids Res., 27(9), 2006-2014), and shown to bind to an invariant element on the surface of a living organism.

The success of the process of in vitro evolution has also been applied to evolve RNAs that contain cis-acting elements that are involved in nuclear transport, nuclear retention and inhibition of export of nuclear RNAs. In contrast to the nucleic acids of the SELEX patents, these RNA sequences were selected by their ability to localize in the nuclei of Xenopus oocytes (Grimm, C. et al (1997) Proc. Natl. Acad. Sci. USA, 94, 10122-10127; Grimm, C., et al (1997) EMBO J., 16(4), 793-798), and were not selected by their informational content nor by their ability to bind specific targets. This work indicates the ability of noninformational nucleic acids to affect their localization within cells.

A desirable approach to solving the above-mentioned problems faced by the pharmaceutical industry would be to select drug candidates for clinical trials based on the in vivo efficacy of the drugs. It would also be useful to modify several drugs simultaneously while selecting them under in vivo conditions. In addition, since at present another difficulty lies in delivering drugs to the inside of cells, it would be desirable to apply this approach to enhance the accessibility of known drugs to the intracellular compartment of cells. Such an approach would yield a greater number of drugs that are effective in vivo in a timesaving and costefficient manner. Furthermore, knowing that nucleic acids can perform functions other than encoding proteins, it would be advantageous to exploit this knowledge in combination with exponential enrichment in vitro technology to identify nucleic acid molecules that localize within cells, and combine them with known drugs ultimately to enhance the efficacy of known drugs by simply increasing drug access into cells.

### SUMMARY OF THE INVENTION

The present invention overcomes many of the limitations of the prior art by providing a new and novel method that exploits in vitro evolution in an in vivo setting for improving the efficacy of known drugs.

In general, the method of the present invention involves administering a large population of oligonucleotides to a biological test system, isolating the oligonucleotides that localize in the intracellular compartment of cells, combining the isolated intracellular oligonucleotide with known drugs to form modified drugs that are chimeric oligonucleotide-drug molecules, and screening in vivo for the chimeric molecules that display an efficacy superior to that of the unmodified drug.

It is an important objective of the present invention to provide a method that applies in vitro evolution to identify intracellular oligonucleotides in vivo and produce an end population of intracellular oligonucleotides that are identified for their ability to enter and to be retained within cells. In the preferred embodiment, the end population of oligonucleotides is combined with known drugs to form chimeric oligonucleotide-drug molecules that in turn are screened in vivo to identify the chimeric molecules that are preferentially retained within the cell. Alternatively, a known drug may first be combined with an initial or subsequent population of intracellular oligonucleotides, and the resulting chimeric molecules are then subjected to iterative rounds of evolution to yield an end population of chimeric molecules that is enriched in the species of chimeric molecules that are preferentially retained within the cells.

Another objective of the present invention is to increase the relevance of the chimeric drug by first isolating a population of intracellular oligonucleotides from cells in culture or from cells isolated from organs of an animal, and then submitting the isolated population of oligonucleotides to additional rounds of selection in human cells. The population of intracellular oligonucleotides that accumulates within the human cells is then identified, including whether that population reaches, reaches a concentration that is equal or greater than that attained in the cultured cells or those isolated from an organ. Preferably, the human intracellular oligonucleotides are identified prior to combining them with a known drug. Alternatively, a population enriched in intracellular oligonucleotides is first obtained in an animal model. Thereafter, a known drug is combined with said oligonucleotide population, and the chimeric molecule is tested for the desired properties in a different biological test system, such as cells of human origin.

Another objective of the present invention is to increase specificity of the selection. For example, if oligonucleotides became concentrated in a desired organ such as the brain, as well as in an organ such as the kidney, where accumulation of oligonucleotides is not desired, selection could be refined by including a negative selection step as follows: after selection, oligonucleotides from brain cells are amplified, and one half of the oligonucleotide population is injected in the animal. Cells from the kidney are isolated and the intracellular oligonucleotides are amplified and used to perform subtractive hybridization with the amplified intracellular oligonucleotides obtained from brain cells. Various methods for performing subtractive hybridization are known in the art.

Another objective of the present invention is to improve the retention of drugs that are typically administered orally, whereby ingested oligonucleotides will be selected according to their ability to reach the circulation. Following oral administration of the initial pool of oligonucleotides, the oligonucleotides that reach the circulation will be isolated from blood components, amplified and, preferably, subjected to subsequent rounds of ingestion, isolation and amplification to yield an end population of oligonucleotides that are more likely to reach organ cells and impart increased cell retention of those drugs that are typically administered orally.

It is another objective of the present invention to further increase the intracellular concentration of the chimeric molecules by mutagenizing the end oligonucleotide population, and testing said mutated oligonucleotide end population for desired properties that are enhanced over those of the non-mutated end population.

### BRIEF DESCRIPTION OF THE DRAWINGS

The present invention will become more fully understood from the detailed description hereinbelow, and together with the accompanying drawings which are provided by way of illustration only, and are not intended to limit the present invention, wherein:

FIG. 1 a-d show schematic examples of chemical reactions of how drug molecules may be covalently attached to the oligonucleotides molecules via linkers.

FIG. 2 shows a schematic representation of a method for producing a chimeric molecule in accordance with the invention, said chimeric molecule having improved cytotoxic properties;

FIG. 3 shows a schematic representation of a method for selecting, in vivo, for sequences, which have universal cancer improving properties.

### DETAILED DESCRIPTION OF THE INVENTION

By determining in vivo the intracellular concentration of modified known drugs, herein referred to as chimeric drug-oligonucleotide molecules, and comparing it to the intracellular concentration reached by unmodified known drugs, the method of the present invention simultaneously screens for three of the most important properties of drugs at the same time, namely drug targeting, drug uptake, and drug retention, thus ensuring that the selection for any one characteristic is not made at the expense of another.

The present invention aims at identifying intracellular oligonucleotides that have desired properties. The desired properties include the ability to access the intracellular compartment of cells, the ability to increase the intracellular concentration of a known drug, and the ability to increase the organ specificity of a known drug, and the inability to specifically bind to target molecules as described below.

The present invention seeks to improve the pharmacological activity of known drugs, wherein pharmacological activity of a drug is usually defined as its ability to inhibit, agonize or antagonize a target by binding with the requisite affinity. Binding is achieved by a stereoelectronic interaction whereby the target of the drug recognizes the three-dimensional arrangement of functional groups and their electron and charge density. Prior biotechnology aims at developing ligands including nucleic acids, amino acids and small organic molecules to explore the three-dimensional “shape space” of their targets and bind to them with high affinity and specificity (Maulik, S. and Patel, S. D. (1997) Molecular Biotechnology. Therapeutic Applications and Strategies. 72-77). In contrast, the present invention aims at increasing the affinity and specificity of already known drug ligands by exploiting the potential three-dimensional arrangement of the drugs to increase the established desired properties of said drugs. In other words, the present invention does not select nucleic acids for their specific binding properties.

In the context of this application the term therapeutic properties encompasses drug specificity, drug uptake, and drug retention, which are all directly related to the specific binding affinity of the drugs for the target. Drug specificity refers to the ability of a drug to target only the desired organ, without affecting other organs where the drug's activity is not desired; drug uptake refers to the ability of a drug to be internalized by cells, and is evidenced by the intracellular concentration of the drug; this property is closely related to the ability of the drug to be retained within the cell, referred to as drug retention. It is well known that cells can actively extrude drugs from the cytoplasm to the extracellular space. An example is the MDR pump present within the cell membrane that is responsible for the extrusion of chemotherapeutic agents, and which readily mutates to confer resistance to multiple drugs.

The present invention seeks to improve these three properties of known drugs by discovering intracellular oligonucleotides, preferably in vivo, that, when combined with a known drug increase the pharmaceutical activity of the drug by imparting increased specificity, retention and uptake, as determined by an increase in the intracellular concentration of the chimeric oligonucleotide-drug molecules when compared to the intracellular concentration of unmodified known drugs.

The intracellular oligonucleotides may also be combined with known drugs to improve the therapeutic index, bioavailability, and the stability of the known drug, as well as the drug's known spectrum of activity, wherein therapeutic index relates to the ratio between the highest and lowest concentration known to have a desired pharmaceutical effect without causing harmful side-effects; bioavailability refers to the ability of a drug to reach a target site without losing its therapeutic properties; stability of a drug includes the drug's resistance to enzymatic degradation, its clearance by the lymphatic and renal systems; and spectrum of activity refers to the drug's ability to produce simultaneously two or more beneficial effects. The properties of the drugs mentioned herein are merely examples, and any person versed in the art of pharmaceutics will be aware of the fact that other improvements in drugs may be desired.

The oligonucleotides of the present invention may be either RNA or DNA oligonucleotides. DNA oligonucleotides are directly synthesized using a DNA synthesizer by methods known in the art. DNA oligonucleotides can also be synthesized to contain the T7 promoter sequence so that they may be used to transcribe the population of RNA oligonucleotides using T7 polymerase. The resulting RNA and DNA oligonucleotides are purified by denaturing polyacrylamide gel electrophoresis, and are eluted in a buffer appropriate for administering the oligonucleotides to a biological test system.

A population of oligonucleotides herein refers to a large that numbers between 10¹⁵ and 10¹⁸ oligonucleotides, wherein the oligonucleotides in the population are between 15 and 80 nucleotide bases long. The oligonucleotides may comprise a sequence that is random, partially random or doped, wherein a partially random sequence comprises a conserved sequence and a random sequence, and a doped sequence is one that typically is between 90 and 95% conserved with the remainder being random. In the preferred embodiment, the oligonucleotides comprise random sequences that are flanked by predetermined sequences which assist in the amplification steps and include primer sequences for amplification by PCR. PCR can also be used to quantify intracellular oligonucleotides by the precess of quantitative PCR. (Holland, P. M. et al (1991) Proc. Natl. Acad. Sci. USA 88, 7276-7280; U.S. Pat. No. 5,210,015; Lee, L. G. et al (1993) Nucleic Acids Res., 21, 3761-3766; Livak, K. J. et al (1995) PCR Methods and Applications, 4, 357-362)

The oligonucleotides may be modified to contain specific primer tags, and nuclease-resistant nucleotides (Beaudry, A. et al (2000) Chem. Biol., 7, 323-334). Nuclease-resistant nucleotides may be added to the 5′ end of the oligonucleotide during the PCR, whereas addition to the 3′ end of the oligonucleotides is performed following PCR by polyA polymerase. Oligonucleotides can also be engineered to contain modifications of the sugar-phosphate backbone, such as by addition of a thiol group to the phosphate. Other methods for modifying oligonucleotides are known to those skilled in the art. The specific primer tag includes a known sequence to which a PCR primer can be annealed. Tags provide a means to identify or recover intracellular oligonucleotides following administrating them to a biological system. Many other tags are available and the methods for including tags with a nucleic acid are well known in the art and kits for the modification/labelling of nucleic acids are readily available from several vendors.

It is presently preferred in accordance with the invention to increase the stability of oligonucleotides by incorporating nuclease resistant oligonucleotides at both their 5′ and 3′ ends.

In one preferred embodiment, an initial population of oligonucleotides is synthesized as described above. This initial population refers to a collection of between 10¹⁵ and 10¹⁸ oligonucleotides which differ from each other in sequence, and which is administered to a biological system at the beginning of the first round of evolution.

A biological system herein includes ex vivo and in vivo biological systems; wherein the ex vivo biological system comprises cells in culture, or an isolated perfused organ, and the in vivo system comprises an animal or a patient. Further, the cell culture may comprise a single type or a plurality of diverse cell types; an isolated perfused organ may be for example an isolated perfused heart, or an isolated perfused kidney; an animal typically comprises smaller laboratory animals such as mice or rabbits, but may include larger species such as primates. The term cells includes eukaryotic and prokaryotic cells.

The term administering to a biological system refers to delivering in vivo a populations of oligonucleotides or chimeric drug-oligonucleotide by any manner known in the art to administer pharmaceutical substances including oral, parenteral, rectal, nasal, topical, and may be formulated as a vaccine composition, together with any pharmaceutically or immunologically acceptable carrier, which may be chosen in accordance with the preferred mode of administration. When the oligonucleotides or chimeric molecules are administered to an ex vivo system, administering simply means adding the oligonucleotides to the medium in which the cells are growing.

Following administration, the oligonucleotides of interest, the intracellular oligonucleotides, are isolated from the intracellular space and identified as described below. Intracellular space includes the cytoplasm, the nucleus, cellular organelles, and other intracellular components.

Intracellular oligonucleotides refers to single-stranded DNA or single-stranded RNA oligonucleotides that are isolated from the intracellular compartment of cells in a biological test system due to their ability to enter cells, following administration of a population of oligonucleotides. The oligonucleotides are isolated by their physical localization within the cell; selection is not biased by other biochemical parameters such as whatever binding affinity the oligonucleotides may have for a three-dimensional target structure. In a presently preferred arrangement, they are selected by their ability to enter cells, and not by their specific binding to target molecules. A three-dimensional target structure herein refers to a structure to which an oligonucleotide has been shown to bind specifically; wherein said target structures are those defined in any one of the SELEX™ patents. This selection criterion, in conjunction with the use of selection in vivo, fundamentally distinguishes the population of oligonucleotides obtained by the method of the present invention versus those nucleic acids that are identified in accordance with the method taught by the prior art including partitioning step required by certain of the prior art is not required in the present invention isolating the oligonucleotides that have entered the cell.

By using conventional oligosynthesis an enormous number of unique molecules, each embodying diverse structure and chemical topography can be created. Within these oligonucelotide populations there will inevitably emerge substantial diversity of molecular structures. The population due to its large diversity, will contain members that will ferry drug payloads into targeted cells. This is true because either the nucleic acid will be able to mimic a molecule that naturally is transported into the target cell or it can optimize certain mechanisms that are known to enhance uptake of DNA in vivo. There are many reports in the literature alluding to a receptor that can transport nucleic acid in vivo, however, because the invention is based on functional assays, knowledge of the exact mechanism by which selected nucleic acids enter into cells is irrelevant.

Isolating the intracellular oligonucleotides means using a method that first separates the cytoplasmic fraction of cells from the cell membrane and intracellular components. The step of isolating intracellular oligonucleotides immediately follows the first step of administering the oligonucleotides to a biological test system. When the biological system is a cell culture, isolating means recovering cells that grow in suspension by known methods of centrifugation, or in the case where the cells grow adherent to the culture dish, isolating means detaching the cells either by trypsinization or EDTA, then collecting them by centrifugation. When the biological system is an animal, an organ or a tissue sample is obtained, the cells are dispersed by enzymatic means known in the art, and the cells are collected by centrifugation. The collected cells are then treated with a proteinase, then disrupted by known means such as hypotonic lysis. Subcellular fractionation is performed to separate nuclei, intracellular membrane fractions, mitochondria, and other organelles from the cytoplasm. Nucleic acids are then extracted from the subcellular fractions using known methods that employ SDS, proteinase K and phenol. Genomic DNA is eliminated by digesting it with endonuclease. The isolation process will preferably be performed at a low temperature between 0 and 4° C. to avoid internalization of lytic enzymes.

The intracellular oligonucleotides are identified by their tag sequence, which is used to amplify them. Following the amplification step of the end population of oligonucleotides, these may be cloned and sequenced.

Amplifying an intracellular oligonucleotide means increasing the number of copies of the population of intracellular oligonucleotides that were isolated as described above. Methods for amplifying DNA and RNA oligonucleotides are well known in the art. Amplification of intracellular DNA oligonucleotides is accomplished preferably by PCR as described above, whereas amplification of intracellular RNA oligonucleotides requires reverse transcription coupled to PCR (RT-PCR), which is then followed by transcription of the cDNA to produce a subsequent population of RNA oligonucleotides to be used in the next round of selection. Multiple cycles of isolation and amplification may be performed as is required to reach an end population of intracellular oligonucleotides.

The end population of oligonucleotides herein refers to a population of oligonucleotides numbering between 10¹⁵ and 10¹⁸ that is enriched in the oligonucleotide species that are selected for their ability to enter cells. Preferably, the end population includes between 5 and 50 species of oligonucleotides, and alternatively this population may be further subjected to additional rounds of selection to include a single oligonucleotide specie. The process of identifying and end populations may require that multiple subsequent population of oligonucleotides be generated. The intracellular oligonucleotides in an end population may be subjected to mutagenizing processes such as mutagenizing PCR, so as to further refine and improve the desired properties of the end population. Methods for altering sequences of oligonucleotides are well known in the art.

A subsequent population of oligonucleotides refers to a population that is intermediate between the initial and end populations described above. The number of subsequent populations varies according to the number of iterative rounds of evolution that are required to reach a desired end population. An end population refers to a population of extracellular oligonucleotides that is sufficiently enriched in one or more oligonucleotide species that display desired properties.

Ultimately, the end population of oligonucleotides is combined with a known drug to enhance the therapeutic properties of said drug. Combining a known drug to an a population of oligonucleotides refers to a process whereby the known drug and the oligonucleotide are attached to each other by formation of covalent bonds, forces. In the preferred embodiment, the drug is attached to the oligonucleotide by formation of covalent bonds between the drug and the oligonucleotide.

Drug molecules used in the molecular evolution will also typically be fluorescent, immunologically or otherwise detectable, to allow detection of molecules in cells and tissue samples. Drug molecules, will be covalently attached to the oligonucleotides molecules via linkers. The chemical linkage will be designed and synthesized to conform to the requirements of the selection process and to therapeutic considerations. The criteria for optimal linkages may vary between applications; some examples are described below.

The most general linkage will be of such length and flexibility as to minimize the interference between the functions of the oligonucleotides and the drug moiety. Such interference may arise if the linked drug affects the proper folding of the oligonucleotides, or if the linkage causes steric hindrance to the binding of either the drug or the oligonucleotides to targets or transporters. Typical linkers may be between 10-20 atoms in length and may consist of aliphatic chains or of chains including amide, ester, etheric, or other bonds and various side groups. An example for such a linker could be poly-(ethylene glycol) (PEG). It has been demonstrated that the use of the extended PEG linker releases steric hindrance of Mab transport vectors on binding of EGF to its cognate receptor on glioma cells (Deguchi, L. Y. et (1999) Bioconjugate Chem., 10, 32-37

The hydrophobicity of the linkage can also be adjusted for different needs. Thus, hydrophilic chains may be used to allow high water solubility and to reduce non-specific absorption or sequestration. On the other hand, more hydrophobic linkers could enhance membrane penetration or sub-cellular localization of the drugs. These properties of the linker can be controlled by modifying the bonds along the chain, as described above, as well as by addition of side groups. For example N-hydroxysulfosuccinimide, is an hydrophilic ligand for the preparation of active esters. Incorporating this ligand into cross-linking reagents such as, 3,3′-dithiobis(sulfosuccinimidyl propionate) and bis(sulfosuccinimidyl) suberate will increase their hydrophilic characters. N-hydroxysulfosuccinimide active esters: bis(N-hydroxysulfosuccinimide) esters of two dicarboxylic acids are hydrophilic, membrane-impermeant, protein cross-linkers. Staros JV. (1982) Biochemistry, 21(17), 3950-3955.

Whereas chemically stable linkers are preferable during the molecular evolution process, in some circumstances a cleavable linkage may be advantageous. For example, a linkage may be designed so that the drug is inactive when bound to the oligonucleotides and become active only after the linkage is cleaved. This will prevent activation of the drug at the wrong location or time. The linkage can be made pH-sensitive, so that it is cleaved only after internalisation into endosomes; it may include an easily reversible disulphide bond, which will be cleaved in the reducing environment of the cell cytoplasm. Linkers possessing a disulfide group such as Ar—S—S—(CH2)n—S—S—Ar are mentioned in the literature and may be synthetized easily (Kliche, W. et al (1999) Biochemistry, 38, 10307-10317). These cross-linkers easily reacted with thiols in a disulfide exchange reaction that proceeded exclusively via the route forming the dialkyl disulfide. In more advanced applications, the linkage could be made cleavable by specific enzymes, such as hydrolases. Again, this could enhance the specificity of drug action, by combining the targeting specificity offered by the oligonucleotides to the activation of the drugs by enzymes that are more active in the target cells. Another possible application is allowing a sequence of events that cannot be achieved in its entirety by drugs permanently attached to the oligonucleotides. For example, a oligonucleotides may lead the drug across the blood-brain barrier, but the drug-oligonucleotides chimera may not effectively enter the target cells. In that case, cleavage of the link may release the drug in a form that may be taken up by the cells.

The actual conjugation of the oligonucleotides, linker and drug molecules may be achieved by a variety of well-known chemistries. Typically, a linker may be incorporated at a terminus of the oligonucleotide during synthesis, in the form of a modified nucleotide precursor.

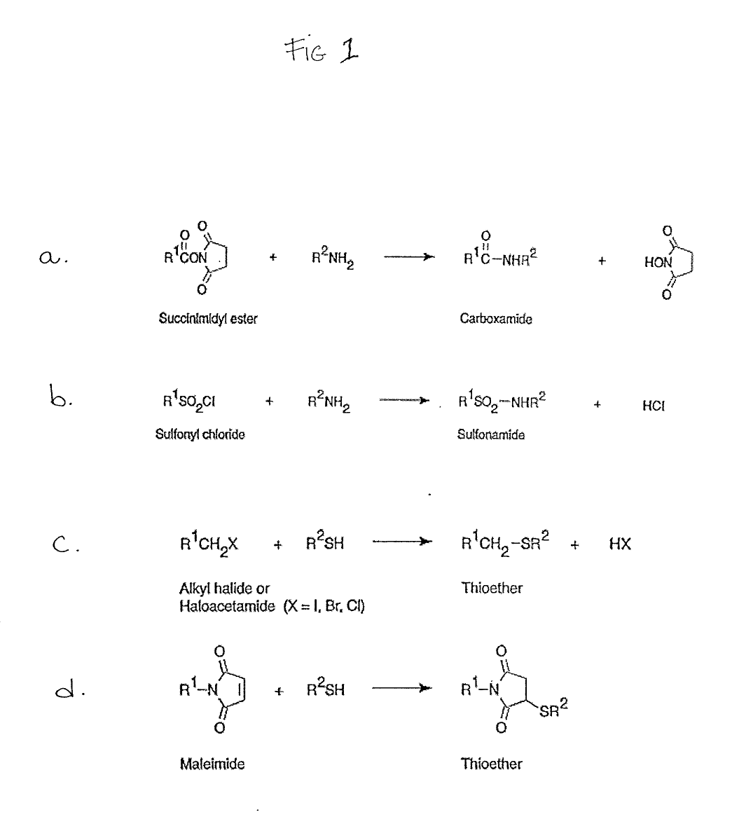

Oligonucleotides bearing an active probe are available commercially or custom made. FIG. 1a shows an example of how one can use an oligonucleotide with a reactive-amine site such as succinimidyl ester which is an excellent reagent for amine modification because the amide bonds they form are as stable as peptide bonds. These reagents also show good reactivity with aliphatic amines and very low reactivity with aromatic amines, alcohols, phenols (including tyrosine) and histidine. This selectivity is important in order to maintain a specific reaction without side reactions that may disturb the final product.

Another group that may be used is Sulfonyl Chloride, which produces the very stable sulfonamide in reaction with a primary amine, as shown in FIG. 1b.

Should amines not be specific enough for some purposes, thiol-reactive sits will be used, as shown in FIG. 1c. The thiol-reactive functional groups are primarily alkylating reagents, including iodoacetamides, maleimides, benzylic halides and bromomethylketones. Arylating reagents such as NBD halides react with thiols or amines by a similar substitution of the aromatic halide. Reaction of any of these functional groups with thiols usually proceeds rapidly at or below room temperature in the physiological pH range (pH 6.5-8.0) to yield chemically stable thioethers. The drug may be linked to a thiol-reactive functional group and the oligonucleotides to a thiol or vice versa (Gaur, R. K. et al (1989) Nucleic Acids Res, 9, 17,4404 PN9567.

Another approach may be indirect crosslinking of the amines on one side and to the thiols in a second. Thiol-reactive groups such as Maleimides or lodoacetamides are typically introduced into the second biomolecule by modifying a few of its amines with a heterobifunctional crosslinker containing a succinimidyl ester and either a maleimide or an iodoacetamide. The maleimide- or iodoacetamide-modified biomolecule is then reacted with the thiol-containing biomolecule to form a stable thioether crosslink (see FIG. 1d). Chromatographic methods are usually employed to separate the higher molecular weight heteroconjugate from the unconjugated biomolecules.

When designing the conjugation chemistry, special care must be applied to prevent loss of activity of the drug, and to assure that the linkage is formed in an efficient and unique manner, with no undesired or uncharacterized side-products.

During the selection cycles used in molecular evolution of oligonucleotidess, the linker and drug moieties will be changed. This is important to ensure that the targeting properties of the chimeric molecules are indeed due to the oligonucleotides, and not specific to the drug and linker. In advanced applications, it may come about the particular linkers confer desirable properties to the chimeras; If such effects are discovered, they will be incorporated in the design of drug-oligonucleotides chimeras.

The combining step produces a plurality of different chimeric molecule species, each specie being different from the other due to the sequence of its oligonucleotide portion. Another diversity of the chimeric molecules may be denoted due to the fact that there will be times when the oligonucleotide may be bound to more than one position of the drug thus further increasing the possible combinations.

The word drug herein refers to any moiety, which may be an organic or inorganic molecule, a protein, peptide or polypeptide, a hormone, a fatty acid, a nucleotide, a polysaccharide, a plant extract, whether isolated or synthetically produced, etc. which is known to have a beneficial therapeutic activity, when administered to a subject, and includes drugs that are used to treat cardiovascular and neurological diseases, as well as cancer, diabetes, asthma, allergies, inflammation, infections and liver disease. Known drugs refers to drugs that are known to have therapeutic effects as well as drugs that have not reached commercial development, such as clinical trials. The present invention would allow to revisit those drugs that are not selected for clinical trials, and improve them by the methods disclosed herein. The cardiovascular drugs include beta-blockers such as metoprolol and carvetilol, phosphodiesterase inhibitors such as milrinone, as well as other drugs including dobutamine and Angiotensin II antagonists. The neurological drugs include those drugs that by their combination with the oligonucleotides of the present invention, will be able to be shunted across the blood-brain barrier, as well as those drugs whose targeting of nerve cells will be improved by the oligonucleotides of the present invention. The cancer drugs include the taxanes such as Paclitaxel and Docetaxel, as well as Tamoxifen, gemcitabine, irinotecan, and cisplatin. In the case of diabetes, insulin is the drug whose efficiency is sought to be improved by the present invention. Asthma and anti-inflammatory drugs include A1 adenosine receptor antagonists. Drugs to treat infection include antibiotics such as penicillin and cephalosporins, as well as new drugs that are currently used to treat M. tuberculosis infections. The oligonucleotides of the present invention may also be combined with monoclonal antibodies that are used in particular to target antigens that are recognized to play a role in various cancers. Applying the present invention to monoclonal antibodies is expected to improve both the selectivity and affinity of the antibody for the antigen, as well as to potentiate the use of monoclonal antibodies to target intracellular antigens.

There exist several standard methods for determining the intracellular concentration of a drug, and the methods should be tailored to each specific drug and its application. Typically, a cell extract is made and clarified of proteins, and the resulting solution is subjected to a quantitative test. The circulating concentration of a drug or a chimeric drug is measured using clarified serum by the same methods used with cell extracts Quantitative tests may be immunological, chromatographic, or be based on the binding properties of the drug to a target in vitro. Methods that can be used to measure the concentration of the chimeric drug as well as the free drug include Mass spectrometry, which is usually used in conjuction with liquid chromatography or high-pressure liquid chromatography (Font, E. et al (1999) 43(12), 2964-2968; Kerns, E. H. et al (1998) Rapid Commun Mass Spectrom (England), 12(10), 620-624), and radio- and enzyme-linked immunoassays (Horton J K; et al (1999) Anal Biochem (United States), 27(1), 18-28; Goujon L; et al (1998) J Immunol Methods (Netherlands), 218(1-2),19-30). Methods that rely on the differences in the chemical properties of the chimeric and free drug can also be used. For example, since the chimeric molecule may be much larger than the free drug, gel filtration could be used. Otherwise, differences in partition between aqueous and various organic phases can be used as a method for separation. In some applications, the drug can be radioactively labelled, and the distribution of radioactivity can be followed. This method also allows to track the kinetic parameters of drug distribution, clearance and metabolism. Methods for determining the circulating concentration of a drug include radio- and enzyme immunoassays (Lelievre E; et al (1993) Cancer Res (United States), 53(15), 3536-40).

Identifying the intracellular oligonucleotides that increase the intracellular concetration of a known drug means including cleaving the linkage between the oligonucleotide and the drug by methods known in the art. For example, by reducing a disulfide bond that is susceptible to reductive cleavage by reducing molecules such as glutathione, thioredoxine, or NADPH.

The probability of finding a drug molecule with optimal traits increases proportionately with the number of molecules that can be assayed. The invention allows for the simultaneous screening of up to 10¹⁸ molecules. This level of molecules represents approximately a trillion-fold increase in screening capability than is possible using conventional high-throughput screens.

Alternate embodiments of the present invention include those described in the Summary of the Invention in the present application, as well as those given below as examples. However, having fully described a preferred embodiment of the invention, those skilled in the art will recognize, given the teachings herein, that equivalents exist which do not depart from the invention.

It may be appreciated by those skilled in the art that the literature describing the laboratory techniques needed to perform the processes of the present invention is extensive, and only exemplary references have been cited. All of the references cited herein are incorporated by reference in their entirety.

### EXAMPLES

The following examples are non-limiting and are given to further elucidate the scope of the present invention.

### Example 1

### Method for Increasing the Pharmacological Activity of Anticancer Drugs.

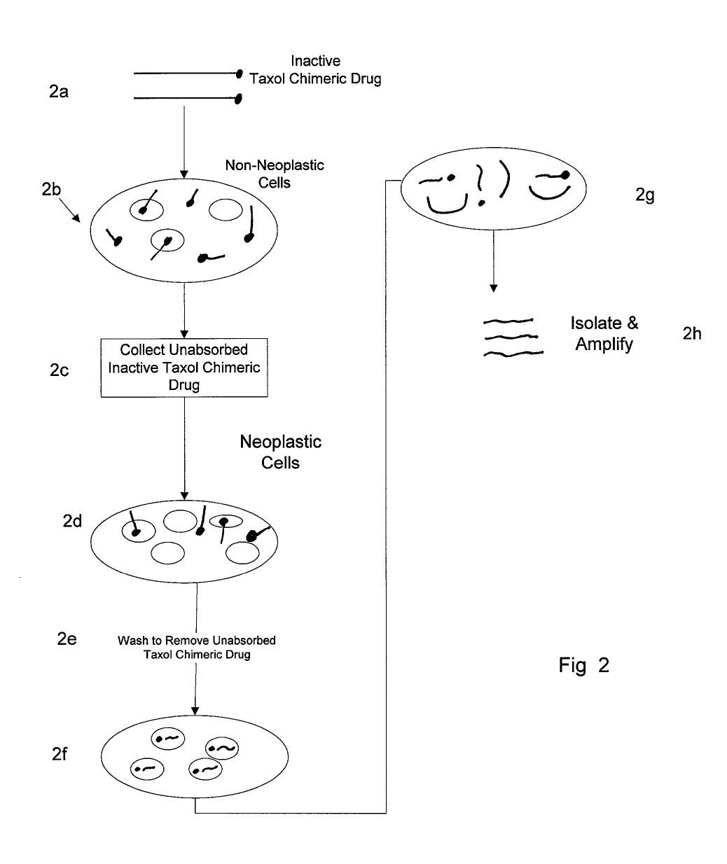

Taxol is an common therapeutic agent used for the treatment of breast cancer, and acts on rapidly proliferating cells. However, one of the problems with Taxol is that it is toxic to all types of actively proliferating cells, whether neoplastic or not. The following example, taken in conjunction with the accompanying FIG. 2, show how the method of the present invention may be used to improve the therapeutic index of Taxol. This method uses a cleavable linkage between the oligonucleotide and the the drug that is cleaved only after the drug is internalized into the internal cell structure which may be, in an exemplary arrangement, the cytoplasm. The oligonucleotide-bound drug is inactive when first administered to the cells, and the activity of the drug is regained only after the drug is internalized to the cell and the linkage between the drug and the oligonucleotide is cleaved.

At step 2a, cytotoxic drug molecules are combined with an intial population of random oligonucleotides to generate an initial population of inactive chimeric cytotoxic drug-oligonucleotide molecules. The chimeric molecules are administered to an ex vivo system of non-neoplastic cells growing in culture (2b). Following administration of the chimeric molecules, those that have not entered the cells are collected (2c). This population of chimeric drugs unabsorbed by normal calls are administered to neoplastic cells (2d). The unabsorbed chimeric molecules are removed (2e). As the inactive chimeric drug enters the cytoplasm, the linkage between the drug and the oligonucleotide is cleaved (2f). An example of such a linkage is an easily reversible disulfide bond, which is cleaved when exposed to the reducing environment of the cytoplasm. Cleavage of the bond allows the drug to regain its cytotoxic activity. The intracellular oligonucleotides are released due to cell death caused by the drug (2g). The released oligonucleotides are isolated and amplified to yield a population of intracellular oligonucleotides that are capable of delivering a cytotoxic drug to the intracellular compartment of neoplastic cells (2h). The steps of combining, administering, isolating and amplifying may be repeated to yield an end population of intracellular oligonucleotides that is enriched in the species of intracellular oligonucleotides that can improve the pharmacological activity of cytotoxic drugs.

### Example 2

### Method for Identifying in Vivo Intracellular Oligonucleotides That Can Improve Uptake, Retention and Specificty of Anti-Cancer Drugs.

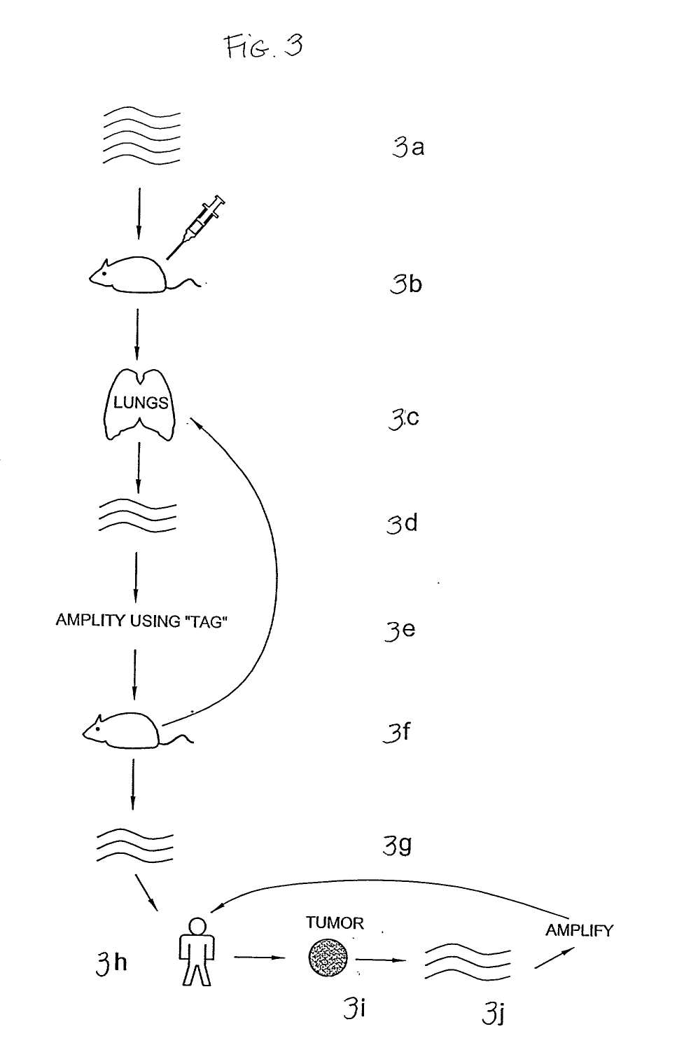

The following method, which may be better understood with reference to FIG. 3, uses in vivo selection to identify intracellular oligonucleotides that preferably localize in the cytoplasm of cancerous cells. The in vivo selection may be carried out first in an animal bearing tumors, and later refined and tested in a patient suffering from a type of cancer. Because the selected intracellular oligonucleotides are isolated from cancerous cells, it is expected that repated cycles of administering, isolating and amplifying intracellular oligonucleotides from these types of cells will yield an end population of intracellular oligonucleotides that will preferably target cancerous cells. These oligonucleotides may in turn be combined with known anticancer drugs to increase the specificty of the drugs.

The first phase of the process is carried out in tumor-bearing animals. An initial oligonucleotide population containing between 10¹⁵ and 10¹⁸ randomly generated oligonucleotide sequence is synthesized using a DNA synthesizer (3a). The population of oligonucleotides is injected intraperitoneally into tumor-bearing experimental animals, for example an animal injected with melanoma, and developing metastasis in the lung (3b, 3c). The melanoma metastasis in the lung is then surgically removed, and the intracellular oligonucleotides from the metastatic cells are isolated and amplified (3d, 3e). This first subsequent population of intracellular oligonucleotides is injected in another animal bearing the same type of tumor as the previous (3f), and steps 3c to 3e are repeated to yield a desired subsequent population of intracellular oligonucleotides that can be further refined and tested in a patient (3g, 3h).

The second phase of the process is carried out in a patient. The desired subsequent population of intracellular oligonucleotides isolated from the tumor-bearing mice, is injected into a consenting cancer patient. Following surgical removal of the tumor, intracellular oligonucleotides are isolated from the cytoplasm of the tumor cells, and are amplified to yield a first ‘human’ population of intracellular oligonucleotides. Said population is then injected into other cancer patients, and the cylce of isolating, amplifying and administering is repeated in other cancer patients too yield an end population of ‘human’ intracellular oligonucleotides wherein preferably one or a few species of oligonucleotides are present.

The intracellular oligonucelotides that are obtained by the process are known to localize in tumor cells, they are known to access the cytoplasm of tumor cells, to be sufficiently resistant to enzymatic degradation, to be retained within the tumor cells, and not to be easily cleared from the body by any physiological process. These intracellular oligonucleotides are prime candidates for improving desired in vivo effects of known anti-cancer agents in humans.

### Example 3

### Method to Eliminate Neoplastic Cell Resistance to An Anticancer Agent.

The detrimental effect of chronic administration of carbamazepine (CBZ) on serum and erythrocyte folates in patients as well as its effects on cultured human ovarian cancer cells SKOV-3 is well documented. In both cases cells become resistant to CBZ. This method uses intracellular carbamazepine-oligonucleotides to overcome neoplastic cell resistance to carbamazepine (CBZ). To obtain CBZ-nucleic acid chimeric molecules that would be less susceptible to neoplastic cell resistance, one can begin selection of intracellular oligonucleotides in CBZ resistant cells.

Intracellular oligonucleotides are selected in vitro for their preferential localization to CBZ-resistant neoplastic cells according to the method of the first example. The end population of intracellular oligonucleotides will contain oligonucleotides that can access CBZ-resistant neoplastic cells. Said oligonucleotides are combined with carbamazepine to yield chimeric carbamazepine-oligonucleotide molecules, and are administered to an animal bearing tumors such as p53 knockout mouse that are receiving CBZ.

Biopsy or sacrifice animal after short interval and then isolate neoplastic cells. Using histological techniques, locate regions of apoptosis within the tumor, isolate the dying cells using the magnetic TAT-bead and method described above, and amplify the intracellular oligonucleotides from the dying cells. After at least 5 cycles of administering, isolating, amplifying and combining, an end population of CBZ intracellular oligonucleotides will be obtained. The oligonucleotides of the end population will be combined with CBZ, and used to target CBZ-resistant tumors.

What is claimed is:

1. A method for identifying intracellular oligonucleotides from an initial population of oligonucleotides having a region of randomized sequence, said method comprising:
a) administering said initial population to a biological system;
b) allowing at least some of the initial population to cross the cell wall and become intracellular oligonucleotides;
c) isolating the intracellular oligonucleotides from the remainder of the initial oligonucleotide population; and
d) amplifying the intracellular oligonucleotides, in vitro, to yield a subsequent population of oligonucleotides that is enriched in the intracellular oligonucleotides, wherein said intracellular oligonucleotides are not oligonucleotides known to have a specific binding affinity for a known three-dimensional structure.

2. The method of 1 wherein the intracellular oligonucleotides are isolated from the intracellular space.

3. The method of claim 1 wherein the biological system is a cell culture.

4. The method of claim 1 wherein the biological test system is a mammal.

5. The method of claim 1 wherein the oligonucleotides of the initial and subsequent populations are modified.

6. The method of claim 5 further comprising the step of:
d) repeating step a) through c) using the subsequent oligonucleotide population of each successive repeat as many times as required to enrich an end population of intracellular oligonucleotides having desired properties.

7. The intracellular oligonucleotides of claim 6.

8. The method of claim 1, 5, or 6 wherein said amplification step employs polymerase chain reaction (PCR).

9. A method for increasing the intracellular concentration of known drugs, comprising:
a) combining a population of oligonucleotides with a known drug to produce chimeric drug-oligonucleotides molecules;
b) administering said chimeric molecules to a biological system;
c) determining the intracellular concentration of the chimeric molecules and comparing the intracellular concentration of said known drug; and
d) identifying the oligonucleotides that increase the intracellular concentration of the known drug.

10. The method of claim 9, wherein said population of oligonucleotides is an initial population.

11. The oligonucleotides of step d of claim 10.

12. The method of claim 9, wherein said population of oligonucleotides is and end population of intracellular oligonucleotides.

13. The oligonucleotides of step d of claim 12.

14. The method of claim 6 wherein the biological system used for identifying intracellular oligonucleotides in a first subsequent population differs from the biological system that is used to identify intracellular oligonucleotides from a second subsequent population.

15. A method for enhancing the tissue specificity of a known anticancer drug, comprising:
a) administering an initial population of oligonucleotides having a region of randomized sequence to cells of a first tumor-bearing mammal;
b) isolating intracellular oligonucleotides from cells of a tumor of said first mammal;
c) amplifying said intracellular oligonucleotides to yield a first subsequent population of intracellular oligonucleotides;
d) administering said first population to cells of a second tumor-bearing mammal;
e) repeating steps b and c to yield a first mammal end population of intracellular oligonucleotides;
f) administering said first end population to the cells of a second tumor-bearing mammal;
g) repeating step e to yield a mammal end population of intracellular oligonucleotides that can be combined with a known anti-cancer drug;
h) combining the mammal end population with a known drug;
i) performing the steps a-d of the method of claim 9.

16. The second mammal end population of intracellular oligonucleotides of claim 15, wherein said first mammal is a mouse and said second mammal is a human.

17. A method for enhancing the efficacy of a known cytotoxic drug, comprising:
a) combining an initial population of oligonucleotides having a region of randomized sequence with a known cytotoxic drug to yield a first population of inactive chimeric drug-oligonucleotide molecules;
b) administering said inactive chimeric population to non-neoplastic cells in culture;
c) collecting the inactive chimeric drug-oligonucleotide molecules that have not entered the cells;
d) administering said inactive chimeric population of step c to neoplastic cells in culture;
e) removing and discarding the chimeric drugs that have not entered the cells;
f) isolating the intracellular oligonucleotides that are released by the cells;
g) amplifying the released oligonucleotides to generate a subsequent population of intracellular oligonucleotides;
h) combining said subsequent population with said cytotoxic drug to yield a subsequent population of inactive chimeric drug-intracellular oligonucleotide molecules;
i) repeating steps b through g to yield an end population of intracellular oligonucleotide molecules.

18. The intracellular oligonucleotides of step i of claim 17.

19. A method for increasing organ specificity of intracellular oligonucleotides, comprising:
a) administering an initial population of oligonucleotides having a region of randomized sequence to an animal;
b) isolating intracellular oligonucleotides in a first and a second organ, wherein first and second organ are different;
c) amplifying the intracellular oligonucleotides of step b to yield a first subsequent population of first organ intracellular oligonucleotides, and a second subsequent population of second organ intracellular oligonucleotides; and
d) identifying first organ intracellular oligonucleotides that are not present in said subsequent population of second organ intracellular oligonucleotides to yield a population of organ-specific intracellular oligonucleotides.

20. The organ-specific intracellular oligonucleotides of claim 19.

21. A method for increasing the targeting of a known drug comprising:
a) combining the organ-specific intracellular oligonucleotides of claim 19 with a known drug;
b). administering said chimeric molecules to a biological system;
c) determining the intracellular concentration of the chimeric molecules and comparing the intracellular concentration of said known drug; and
d) identifying the oligonucleotides that increase the intracellular concentration of the known drug.

21. The oligonucleotides identified by the method of claim 20.
